(19) United States
(12) Patent Application Publication
(10) Pub. No.: US 2004/0096470 A1
(43) Pub. Date: May 20, 2004

(54) Cosmetic composition

(76) Inventors: Kojo Tanaka, Higashinada-ku (JP); Chie Kawano, Yukuhashi (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/384,438

(22) Filed: Mar. 7, 2003

Related U.S. Application Data
(60) Provisional application No. 60/363,625, filed on Mar. 12, 2002.

(51) Int. Cl. A61K 7/021; A61K 7/00

(52) U.S. Cl. 424/401; 424/63

(57) ABSTRACT

Disclosed is a cosmetic composition comprising: (a) a double coated pigment coated by a mixture of a fluorine coating compound and a silicone coating compound; (b) a light silicone oil having a viscosity of no more than about 60 mPas and a volatility as such that not more than 35% evaporates after standing at 150° C. at normal pressure for 24 hours; and (c) a gelling agent.

### CROSS-REFERENCE TO RELATED APPLICATIONS

This application claims the benefit of priority to U.S. Provisional Application Serial No. 60/363,625, filed Mar. 12, 2002, which is herein incorporated by reference.

### FIELD OF THE INVENTION

The present invention relates to cosmetic compositions which contain a double coated pigment coated by a mixture of fluorine material and silicone material; a light silicone oil; and a gelling agent.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions in the form of solids, such as loose powders and pressed powders are popular among consumers who enjoy the fresh light feeling on the skin. Solid foundations packaged in compacts are particularly suitable, as such products can be carried for use.

Cosmetic compositions containing fluorine coated pigments and fluorine/silicone double coated pigments are known, such as in JPA 3-264511, JPA 5-58838, JPA 4-225075, JPA 9-301827, JPA 11-310517, JPA 2000-212024, JPA 2000-256133, JPA 2001-302455, and JPA 2001-316223. Fluorine coating is known in the art to provide a pigment good resistance against water and sebum, thereby, fluorine coated pigments are used in cosmetic compositions for providing shine control and good wear resistance over a period of time. While fluorine coating and/or fluorine/silicone coating can provide such advantages, it has also been found that pigments so treated may have less than sufficient wettability with the oil binder, and thus can result in decreased adhesion to the skin when applied. Use of various oil binders have been suggested to overcome this shortcoming, however, such suggestions have not been completely satisfactory.

Meanwhile, the use of gelling agents have been known in the art for product forms such as emulsions and that have relatively high liquid oil levels, such as in JPA 8-277302, JPA 60-75405, JPA 7-138129, JPA 11-255616, JPA 2000-229816, JPA 2000-247834, and JPA 279040.

Based on the foregoing, there is a need for a cosmetic composition which has balanced benefits in terms of shine control, transfer resistance, color stability, spreadability when applying on the skin, good adhesion on the skin, and fresh light feel on the skin.

None of the existing art provides all of the advantages and benefits of the present invention.

### SUMMARY

The present invention is directed to a cosmetic composition comprising:

(a) a double coated pigment coated by a mixture of a fluorine coating compound and a silicone coating compound;

(b) a low-volatile silicone oil having a viscosity of no more than about 60 mPas and a volatility as such that not more than 35% evaporates after standing at 150° C. at normal pressure for 24 hours; and

(c) a gelling agent.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All cited references are incorporated herein by reference in their entireties. Citation of any reference is not an admission regarding any determination as to its availability as prior art to the claimed invention.

Herein, “comprising” means that other elements which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Double Coated Pigment

The present composition comprises a double coated pigment component, the double coated pigment being coated by a mixture of a fluorine coating compound and a silicone coating compound. The double coated pigment herein provides shine control, good wear resistance, and color stability. Preferably, the weight percentage of the coating compound mixture, namely the sum of the fluorine coating compound and silicone coating compound, is from about 1% to about 30% of the double coated pigment, more preferably from about 2% to about 25% of the double coated pigment. It is believed that such coating provides a double coated pigment that has balanced benefits in terms of shine control and good wear resistance, as well as wettability with the light silicone oil described hereunder.

Useful fluorine coating compounds herein include diethanolamine salts of perfluoroalkyl phosphate, a highly preferred material with tradename ASAHI GUARD AG530 available from Asahi Glass.

Useful silicone coating compounds herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Highly preferred is n-octyl triethoxy silane with tradename DYNASYLAN OCTEO available from Degussa.

Pigments suitable for such coating can be any pigment that is cosmetically acceptable, such as those exemplified under the section “ADDITIONAL PIGMENTS” hereunder.

Particularly suitable coating compounds for coating the double coated pigments herein include diethanolamine salts of perfluoroalkyl phosphate & dimethicone, diethanolamine salts of perfluoroalkyl phosphate & Acryl copolymer, and diethanolamine salts of perfluoroalkyl phosphate & triethoxycaprylsilane. Commercially available double coated pigments highly useful herein include: talc coated with C₉₋₁₅ fluoroalcohol phosphates and triethoxycaprylylsiloxane by the tradename of FOTS-TALC JA-46R, mica coated with C₉₋₁₅ fluoroalcohol phosphates and triethoxycaprylylsiloxane by the tradename of FOTS MICA Y-3000 and sericite coated with C₉₋₁₅ fluoroalcohol phosphates and triethoxycaprylylsiloxane by the tradename of FOTS-52 SERICITE FSE, available from Daito Kasei, Osaka Japan.

Preferably, the present composition comprises by weight from about 1% to about 99% of the double coated pigment.

In one preferred embodiment, the composition is a powder foundation for the facial skin. In such preferred embodiment, the composition preferably comprises from about 10% to about 80%, more preferably from about 20% to about 70% of the double coated pigment.

In another preferred embodiment, the composition is a foundation having lower levels of pigments. In such preferred embodiment, the composition preferably comprises from about 1% to about 40% of the double coated pigment.

Light Silicone Oil

The present composition comprises by weight from about 0.1% to about 60%, preferably from about 1% to about 30%, more preferably from about 1% to about 15%, still preferably from about 3% to about 10% of a light silicone oil. By light silicone oil herein, what is meant is that the light silicone oil has a viscosity of less than about 60 mPas, and a volatility as such that not more than 35% of the light silicone oil evaporates after standing at 150° C. at normal pressure for 24 hours. Such light silicone oils are believed to enhance the fresh and light feel when the composition is applied to the skin, even when gelling agents, which are generally known to provide a sticky heavy feel, is used in the composition. The light silicone oil further acts as an effective binder for the double coated pigments.

Light silicone oils useful herein also include polyalkyl or polyaryl siloxanes with the following structure (I)

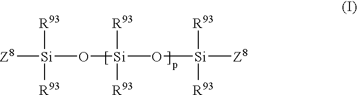

wherein R⁹³ is alkyl or aryl, and p is an integer from about 7 to about 100. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. Polyalkylaryl siloxane fluids can also be used and include, for example, polymethylphenylsiloxanes.

Commercially available herein include methylphenyl polysiloxane having a viscosity of about 16 mPas with tradename KF56 available from ShinEtsu Chemical Co., Ltd., SF 1075 METHYL PHENYL FLUID available from the General Electric Company, 556 COSMETIC GRADE FLUID available from Dow Corning, and polydimethylsiloxane having less than 50 mPas with tradenames SH200 available from Dow Corning, VISCASIL and SF96 series available from the General Electric Company, and KF96 available from ShinEtsu Chemical Co.

Gelling Agent

The present composition comprises by weight from about 0.01% to about 10%, preferably from about 0.05% to about 5%, more preferably from about 0.1% to about 2% of a gelling agent. The gelling agent useful herein are those that are cosmetically acceptable and enhance the binding of the double coated pigments. By the use of gelling agents, the adhesion of the composition to the skin is enhanced, and further, wear resistance is improved.

Gelling agents useful herein include saccharide fatty acid esters, metal soaps, organically modified clay minerals, and mixtures thereof. Specifically, gelling agents useful herein include saccharide fatty acid esters such as dextrin palmitate, sucrose palmitate, metal soap such as aluminium stearate, and magnesium stearate; and organically modified clay minerals such as dioctadecyl dimethyl ammonium modified montmorillonite, dihexadecyl dimethyl ammonium modified montmorillonite. Particularly useful are those which are thixotropic. Thixotropy of the gelling agent is believed to provide good spreadability to the composition when shear is applied for spreading the composition on the skin. Highly preferred herein are thixotropic dextrin fatty acid esters which have an average sugar repeating unit of 3-150, a fatty acid of 8-22 carbons, and a replacement of fatty acid per glucose of 1-3.

Commercially available gelling agents highly useful herein include dextrin palmitate/ethylhexanoate with tradename RHEOPEARL TT available from Chiba Flour Milling Co. Ltd., Chiba Japan.

Additional Binder Material

The present composition may further comprise an additional binder material for providing additional usage benefits and skin feel benefits. The additional binder material herein are those which help pigments, in general, from scattering upon use and carriage. The amount and type of binder is selected depending on the desired characteristic of the product, for example, product form such as loose powder or compact powder, coverage, adhesion to the skin, and various skin feel. The additional binder material herein may also be used for dissolving the gelling agent prior to mixing with other components.

In one preferred embodiment, the cosmetic composition is a powder foundation for the facial skin. In such preferred embodiment, the sum of the light silicone oil, the gelling agent, and the additional binder material amount to from about 1% to about 25% of the composition.

In another preferred embodiment, the composition is a foundation having lower levels of pigments. In such preferred embodiment, the sum of the light silicone oil, the gelling agent, and the additional binder material amount to from about 60% to about 99% of the composition.

Additional binder material useful herein are various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof. Still other oils useful as additional binder material herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oi., rice bran oil, china paulownia oi., Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, cholesteryl derivatives such as cholesteryl 12-hydroxystearate, cholesteryl macadamiate, cholesteryl stearate, and mixtures thereof. Commercially available oils include, for example, tridecyl isononanoate with tradename CRODAMOL TN available from Croda, HEXALAN available from Nisshin Seiyu, tocopherol acetates available from Eisai, cholesteryl 12-hydroxystearate with tradename SALACOS HS available from Nisshin Oil Mills, Ltd., and cholesteryl macadamiate with tradename YOFCO MAC available from Nippon Fine Chemical Co., Ltd.

A wide variety of oils having UV protecting benefit are suitable for use herein, including those which are typically called UV protecting agents. Preferred among those UV protecting agents are those selected from 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-paminobenzoate, p-aminobenzoic acid, 2-phenylbenzimidazole-5-sulfonic acid, octocrylene, oxybenzone, homomenthyl salicylate, octyl salicylate, 4,4′-methoxy-tbutyldibenzoylmethane, 4-isopropyl dibenzoylmethane, 3-benzylidene camphor, 3-(4-methylbenzylidene) camphor, and those by tradenames EUSOLEX 6300, OCTOCRYLENE, PARSOL 1789. These oils can be selected for providing a desired Sun Protection Factor (SPF). SPF is a commonly used measure of photoprotection of a sunscreen against erythema. See Federal Register, Vol. 43, No. 166, pp. 38206-38269, Aug. 25, 1978.

Additional Pigments

The composition may further comprise additional pigments. Additional pigments useful herein include those which are treated or untreated, and are selected depending on the desired characteristic of the product, for example, shade, coverage, UV protection benefit, and various skin feel. Herein, powders that do not necessary color the skin may be included.

In one preferred embodiment, the cosmetic composition is a powder foundation for the facial skin. In such preferred embodiment, the composition further comprises, by weight of the entire composition, from about 4% to about 75% of a base powder other than the double coated pigment, from about 1% to about 30% of a spherical powder other than the double coated pigment, from about 2% to about 15% of a titanium dioxide other than the double coated pigment or spherical powder; and from about 1% to about 10% of a coloring powder other than the double coated pigment, wherein the sum of the double coated pigment and the additional pigments amount to from about 75% to about 99% of the composition.

In another preferred embodiment, the composition is a foundation having lower levels of pigments. In such preferred embodiment, the composition further comprises, by weight of the entire composition, from about 1% to about 40% of a base powder other than the double coated pigment, from about 1% to about 30% of a spherical powder other than the double coated pigment, from about 2% to about 15% of a titanium dioxide other than the double coated pigment or spherical powder; and from about 1% to about 10% of a coloring powder other than the double coated pigment, wherein the sum of the double coated pigment and the additional pigments amount to from about 1% to about 40% of the composition.

Additional pigments useful herein include spherical powders which have a particle size of from about 10 μm to about 50 μm, as well as those which have a particle size of from about 1 μm to about 10 μm. The use of these two different sizes of spherical powders are particularly preferred, as it provides balanced benefits in terms of spreadability when applying on the skin, good adhesion on the skin, and fresh light feel on the skin.

The spherical powders herein can be made of any material that is cosmetically acceptable. Unlimited examples of materials useful for making the large spherical powder and the small spherical powder are; polyacrylates, silicates, sulfates, alumina, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as polymethyl methacrylate and nylon, cross linked polymethyl methacrylate; silicates such as calcium silicate, magnesium silicate, barium silicate, aluminium silicate and silica beads; alumina; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin and solid silicone elastomers. Highly preferred materials for making the spherical powder are polymethyl methacylate.

Commercially available large and small spherical powders highly useful herein include polymethyl methacylate with tradename GANZ PEARL series available from Ganz Chemical Co., Ltd., and SYLYSIA series available from Fuji Sylysia Chemical, Nylon-12 with tradename NYLON POWDER series available from Toray Dow Corning, vinyl dimethicone/methicone silsesquioxane crosspolymer with tradenames KSP series available from ShinEtsu Chemical Co., Ltd., Tokyo Japan, and hardened polyorgano siloxane elastomers with tradenames TREFIL series available from Toray Dow Corning.

Useful additional pigments herein are base powders, including: clay mineral powders such as talc, mica, sericite, silica, magnesium silicate, synthetic fluorphlogopite, calcium silicate, aluminum silicate, bentonite and montomorilonite. The coloring powders useful for additional pigments herein include pearl pigments such as alumina, barium sulfate, calcium secondary phosphate, calcium carbonate, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, cellulose, 12-nylon, 6-nylon, styrene-acrylic acid copolymers, poly proprylene, vinyl chloride polymer, tetrafluoroethylene polymer, boron nitride, fish scale guanine, laked tar color dyes, and laked natural color dyes. Such base powders, titanium dioxides, and coloring powders may be treated with a hydrophobical treatment agent, including: silicone such as Methicone, Dimethicone and perfluoroalkylsilane; fluorine such as diethanolamine salts of perfluoroalkyl phosphate, fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

Additional Components

The composition of the present invention may include other additional components, which may be selected by the artisan according to the desired characteristics of the final product and which are suitable for rendering the composition more cosmetically or aesthetically acceptable or to provide them with additional usage benefits.

Surfactants and water may be included for providing solid compositions that are emulsified, preferably water-in-oil emulsions. Surfactants alone may also provide a binding function. The type and amount of surfactant is selected depending on the nature of the composition.

In one preferred embodiment, the composition is an emulsified foundation. In such preferred embodiment, the sum of the light silicone oil, the gelling agent, the additional binder material, water, and surfactant amount to from about 60% to about 99% of the composition.

The surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename CRILL 6 available from Croda, and sorbitan sesquioleate with tradename ARLACEL 83 available from Kao Atras.

The surfactant can be a silicone-type surfactant. Silicone-type surfactants useful herein are (i), (ii), as shown below, and mixtures thereof.

(i), dimethicone copolyols having the structure:

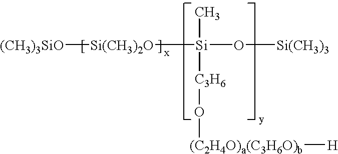

wherein x is an integer from 5 to 100, y is an integer from 1 to 50, a is zero or greater, b is zero or greater, the average sum of a+b being 1-100.

(ii) dimethicone copolyols having the structure:

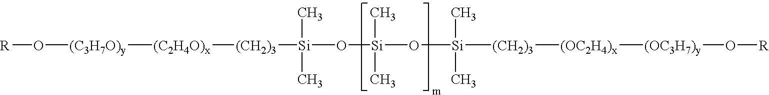

wherein R is selected from the group consisting of hydrogen, methyl, and combinations thereof, m is an integer from 5 to 100, x is independently zero or greater, y is independently zero or greater, the sum of x+y being 1-100.

Commercially available silicone-type surfactants are, for example, DC5225C, BY22-012, BY22-008, SH3746M, SH3771M, SH3772M, SH3773M, SH3775M, SH3748, SH3749, and DC5200, all available from Dow Corning.

Other components which can be formulated into the compositions of the present invention are; preservatives such as benzyl alcohol, methyl paraben, propyl paraben, sodium dehydro acetate, niacinamide, imidazolidinyl area, and EDTA and its salts, perfumes, ultraviolet and infrared screening and absorbing agents, and others.

Method of Preparation and Method of Use

The cosmetic composition of the present invention is useful for various cosmetic products, particularly solid cosmetic products, such as loose powders, powder foundations, emulsion foundations, pressed foundations, blushers, eyeshadows, eyebrow cakes, body powders, and other solid forms of cosmetic products. What is meant by “solid” is that the composition is solid and non-flowing at 25° C.

The cosmetic composition of the present invention may be made by a method well known in the art. When additional binder material is included, the cosmetic composition is preferably made by the steps of:

1) mixing the pigments, including the double coated pigment component, by a mixer;

2) separately dissolving the gelling agent in the additional binder material;

3) mixing the product of step 2) with the light silicone oil; and

4) adding the product of step 1) into the product of step 3) and mixing by a mixer.

When the cosmetic composition is an emulsified composition further comprising water and surfactant, the composition is preferably made by the steps of:

1) mixing the pigment component, including the double coated pigment component, by a mixer;

2) separately dissolving the gelling agent in the additional binder material;

3) mixing the product of step 2) with the light silicone oil and surfactant;

4) adding the product of step 3) into the product of step 1); and

5) adding water into the product of step 4) to effect an emulsification.

Heat may be applied to melt components that are solid at room temperature, or for facilitating homogeneous mixing. When heat is applied, the obtained composition is allowed to cool to room temperature. The obtained composition is placed in a tray container, and pressed as necessary. The tray container is placed in adequate packaging suitable for the user, preferably a compact with a mirror and an applicator for applying the solid composition on the skin. A hermetical packaging may be used as necessary.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-5

The following cosmetic compositions are formed by the process described herein:

Compositions

| No. | Component | 1 | 2 | 3 | 4 | 5 |
|---|---|---|---|---|---|---|
| 1 | Sericite coated with C9-15 fluoroalcohol | 25 | 35 | 35 | 25 | 7 |
| | phosphates and triethoxycaprylylsilane *1 | | | | | |
| 2 | Mica coated with C9-15 fluoroalcohol | 15 | | | 15 | 5 |
| | phosphates and triethoxycaprylylsilane *2 | | | | | |
| 3 | Talc coated with C9-15 fluoroalcohol | | 31.1 | 21.1 | 8.1 | 6 |
| | phosphates and triethoxycaprylylsilane *3 | | | | | |
| 4 | Titanium Dioxide coated with C9-15 | | 12 | | 12 | 7 |
| | fluoroalcohol phosphates and | | | | | |
| | triethoxycaprylylsilane *4 | | | | | |
| 5 | Talc coated with Methicone *5 | 19.49 | | | | |
| 6 | Polymethyl methacrylate 20 μm *6 | 3 | 1 | 2 | 1 | 1 |
| 7 | Polymethyl methacrylate 8 μm *7 | 8 | 3 | 6 | 3 | |
| 8 | Polymethyl methacrylate 6 μm *8 | | 6 | 12 | 6 | 1 |
| 9 | Nylon-12 5 μm *9 | 2 | | | | |
| 10 | Mica coated with Titanium Dioxide *10 | | | | 15 | 1 |
| 11 | Titanium Dioxide coated with Methicone | 10.5 | | 12 | | |
| | *11 | | | | | |
| 12 | Methylparaben *12 | 0.3 | 0.3 | 0.3 | 0.3 | 0.2 |
| 13 | Propylparaben *13 | 0.1 | 0.1 | 0.1 | 0.1 | 0.25 |
| 14 | Iron Oxide coated with Methicone *14 | 2 | 2.5 | 2.5 | 2.5 | 1.5 |
| 15 | Methylphenyl polysiloxane *15 | 8.5 | | | 2.39 | 5 |
| 16 | Dimethicone *16 | | 4.8 | 4 | 5 | 5 |
| 17 | Cyclopentasiloxane *17 | | | | | 22.54 |
| 18 | Dimethicone copolyol & Cyclomethicone & | | | | | 4.5 |
| | Water & Sodium Acetate *18 | | | | | |
| 19 | Dextrin palmitate/ethylhexanoate *19 | 0.1 | 0.1 | 0.5 | 0.1 | 2 |
| 20 | Sorbitan monoisostearate *20 | 1 | | | 0.5 | 2 |
| 21 | D-delta-tocopherol *21 | 0.01 | | | 0.01 | 0.01 |
| 22 | Ethylhexyl Methoxycinnamate *22 | 5 | 4 | 4 | 4 | 9 |
| 23 | Cholesteryl 12-hydroxystearate *23 | | 0.1 | 0.5 | | |
| 24 | Deionized Water | | | | | 20 |

Definitions of Components
*1 Sericite coated with C9-15 fluoroalcohol phosphates and triethoxycaprylylsilane: FOTS SERICITE FSE available from DAITO KASEI KOUGYOU CO., LTD.
*2 Mica coated with C9-15 fluoroalcohol phosphates and triethoxycaprylylsilane: FOTS MICA Y-3000 available from DAITO KASEI KOUGYOU CO., LTD
*3 Talc coated with C9-15 fluoroalcohol phosphates and triethoxycaprylylsilane: FOTS TALC JA-46R available from DAITO KASEI KOUGYOU CO., LTD
*4 Titanium Dioxide coated with C9-15 fluoroalcohol phosphates and triethoxycaprylylsilane: FOTS TiO2 CR-50 available from DAITO KASEI KOUGYOU CO., LTD
*5 Talc coated with Methicone: SI TALC available from MIYOSHI KASEI, INC.
*6 Polymethyl methacrylate: GANZ PEARL GM-2000 available from GANZ CHEMICAL CO., LTD
*7 Polymethyl methacrylate: GANZ PEARL GM-0800S available from GANZ CHEMICAL CO., LTD.
*8 Polymethyl methacrylate: GANZ PEARL GM-0600 available from GANZ CHEMICAL CO., LTD
*9 Nylon-12: NYLON POWDER SP-500 available from TORAY
*10 Mica coated with Titanium Dioxide: FLAMENCO SUPER PEARL available from THE MEARL
*11 Titanium Dioxide coated with Methicone: SI TITANIUM DIOXIDE IS available from TOSHIKI PIGMENT CO., LTD.
*12 Methylparaben: METHYL PARABEN available from UENO PHARMACEUTICALS
*13 Propylparaben: PROPYL PARABEN available from UENO PHARMACEUTICALS
*14 Iron Oxide coated with Methicone: IRON OXIDE series available from DAITO KASEI KOUGYOU CO., LTD.
*15 Methylphenyl polysiloxiane: KF56 available from SHINETSU CHEMICAL CO., LTD.
*16 Dimethicone: SH200 available from Dow Corning
*17 Cyclopentasiloxane: SILICONE DC-245 available from Dow Coming
*18 Dimethicone copolyol & Cyclomethicone & Water & Sodium Acetate: SILICONE DC-5225C available from Dow Corning
*19 Dextrin palmitate/ethylhexanoate: RHEOPEARL TT available from CHIBA FLOUR MILLING CO., LTD.
*20 Sorbitan monoisostearate: CRILL 6 available from CRODA JAPAN KK
*21 D-delta-tocopherol: D-DELTA-TOCOPHEROL available from EISAI CO., LTD.
*22 Ethylhexyl Methoxycinnamate: PARSOL MCX available from ROCHE VITAMINS JAPAN K.K.
*23 Cholesteryl 12-hydroxystearate: SALACOS HS available from Nisshin Oil Mills

Method of Preparation

The cosmetic compositions of Examples 1-4 are prepared as follows: Component numbers 1-14 are mixed by a mixer to make a pigment mixture. Separately, component numbers 21-23 are mixed with the aid of heat, component numbers 19 and 20 are added and mixed until dissolving well, and component numbers 15 and 16 are added and mixed well. The obtained mixture is added to the pigment mixture and mixed by a mixer. The obtained composition is pressed in a tray and set into a compact.

The cosmetic composition of Example 5 is prepared as follows: Component numbers 1-14 are mixed by a mixer to make a pigment mixture. Separately, component numbers 21-23 are mixed with the aid of heat, component numbers 19 and 20 are added and mixed until dissolving well, and component numbers 15-18 are added and mixed well. The pigment mixture is added and mixed by a mixer. Finally component number 24 is added and strongly mixed by a mixer. The obtained composition, which is liquid at elevated temperature, is poured in a tray to cool and solidify. The finally obtained composition is set into a compact.

These embodiments represented by the previous examples are useful as solid foundation products. When applied on the facial skin, they provide many advantages. For example, they can provide balanced benefits in terms of shine control, transfer resistance, color stability, spreadability when applying on the skin, good adhesion on the skin, and fresh light feel on the skin.

What is claimed is:

1. A cosmetic composition comprising:
(a) a double coated pigment coated by a mixture of a fluorine coating compound and a silicone coating compound;
(b) a light silicone oil having a viscosity of no more than about 60 mPas and a volatility as such that not more than 35% evaporates after standing at 150° C. at normal pressure for 24 hours; and
(c) a gelling agent.

2. The cosmetic composition of claim 1 comprising by weight:
(a) from about 1% to about 99% of the double coated pigment;
(b) from about 0.1% to about 60% of the light silicone oil; and
(c) from about 0.01% to about 10% of the gelling agent.

3. The cosmetic composition of claim 1 wherein the fluorine coating compound is selected from diethanolamine salts of perfluoroalkyl phosphate, and wherein the silicone coating compound is selected from the group consisting of methyl polysiloxane, methyl hydrogen polysiloxane, methyl phenyl polysilxoane, n-octyl triethoxy silane, methyl-alpha-styrene polysiloxane, acryl silicone copolymer, and mixtures thereof.

4. The cosmetic composition of claim 1 wherein the weight percentage of the mixture is from about 1% to about 30% of the double coated pigment.

5. The cosmetic composition of claim 1 wherein the gelling agent is selected from the group consisting of saccharide fatty acid esters, metal soaps, organically modified clay minerals, and mixtures thereof.

6. The cosmetic composition of claim 1 wherein the gelling agent is thixotropic.

7. The cosmetic composition of claim 5 wherein the gelling agent is selected from dextrin fatty acid esters.

8. The cosmetic composition of claim 6 wherein the gelling agent is thixotropic.

9. The cosmetic composition of claim 1 further comprising an additional binder oil.

10. A method of making the cosmetic composition of claim 9 wherein the gelling agent is dissolved in the additional binder oil prior to mixing with other components.

11. The cosmetic composition of claim 1 comprising by weight:
(a) from about 10% to about 80% of the double coated pigment;
(b) from about 4% to about 75% of a base powder other than the double coated pigment;
(c) from about 1% to about 30% of a spherical powder other than the double coated pigment;
(d) from about 2% to about 15% of a titanium dioxide other than the other than the double coated pigment or the spherical powder;
(e) from about 1% to about 10% of a coloring powder other than the double coated pigment;
(f) from about 0.1% to about 30% of the light silicone oil;
(g) from about 0.01% to about 10% of the gelling agent; and
(h) an additional binder oil;
wherein the sum of components (a) through (e) amount to from about 75% to about 99% of the composition; and the sum of components (f) through (h) amount to from about 1% to about 25% of the composition.

12. The cosmetic composition of claim 1 comprising by weight:
(a) from about 1% to about 40% of the double coated pigment;
(b) from about 1% to about 40% of a base powder other than the double coated pigment;
(c) from about 1% to about 30% of a spherical powder other than the double coated pigment;
(d) from about 2% to about 15% of a titanium dioxide other than the other than the double coated pigment or the spherical powder;
(e) from about 1% to about 10% of a coloring powder other than the double coated pigment;
(f) from about 0.1% to about 60% of the light silicone oil;
(g) from about 0.01% to about 10% of the gelling agent; and
(h) an additional binder oil;
wherein the sum of components (a) through (e) amount to from about 1% to about 40% of the composition; and the sum of components (f) through (h) amount to from about 60% to about 99% of the composition.

13. The cosmetic composition of claim 12 further comprising by weight:
(i) from about 5% to about 30% of water; and
(j) from about 0.1% to about 10% of a surfactant;
wherein the sum of components (f), (g), (h), (i) and (j) amount to from about 60% to about 99% of the composition.
